(19) United States
(12) Patent Application Publication
(10) Pub. No.: US 2018/0010974 A1
(43) Pub. Date: Jan. 11, 2018

(54) PRESSURE SENSOR SYSTEM

(71) Applicant: Millar Inc., Houston, TX (US)

(72) Inventors: Kenneth M. Bueche, Friendswood, TX (US); David Budgett, Auckland (NZ); Rick Thornton, Bacliff, TX (US); Gabriel P. Kern, Houston, TX (US); Daniel McCormick, Waiheke Island (NZ)

(73) Assignee: Millar Inc., Houston, TX (US)

(21) Appl. No.: 15/643,922

(22) Filed: Jul. 7, 2017

Related U.S. Application Data
(60) Provisional application No. 62/360,093, filed on Jul. 8, 2016.

(51) Int. Cl. G01L 9/00; G01L 13/02; A61B 5/145; G01L 15/00; G01L 19/00; G01L 19/14

(52) U.S. Cl. CPC G01L 9/0054; G01L 19/0069; G01L 9/0041; G01L 19/141; A61B 5/145; G01L 15/00; G01L 13/025; H01L 2224/73265; H01L 2924/10253

(57) ABSTRACT

A pressure sensor system with at least two absolute pressure sensors can have an external sensor with a pressure sensitive surface in contact with atmospheric pressure (proximal) and internal sensors each with a pressure sensitive surface in contact with one or more regions at an unknown pressure (distal). The unknown pressure is determined by a means to calculate the difference between the first sensor and the internal sensors.

### CROSS-REFERENCE TO RELATED APPLICATIONS

This application claims priority to U.S. Provisional Application No. 62/360,093, filed Jul. 8, 2016, which is hereby incorporated by reference.

### FIELD

The disclosure relates generally to medical devices. The disclosure relates specifically to pressure sensors for insertion within a body.

### BACKGROUND

The human body is comprised of various organs that generate, or are subject to, a variety of pressures. These pressures are primarily induced externally due to gravity and include atmospheric compression and body weight opposition. However, there are also a wide range of pressures produced within the body itself. These pressures include those generated by the cardiovascular system, urinary system, digestive tract, musculoskeletal system, central nervous system, and osmotic cell pressures, among others. Most of these pressures are critical for proper health and must be precisely regulated. Blood pressure of the cardiovascular system and cerebral spinal fluid of the central nervous system are two such components that must be regulated to maintain a good state of health. Clinical experience has determined that intracranial pressure should be within the range of 5 to 15 mmHg, and a pressure exceeding 20 mmHg requires urgent medical intervention. The ability to continuously monitor these pressures would allow for early detection and intervention in the event auto-regulation becomes impaired. Even knowing that a particular pressure parameter is increasing would provide useful information to help manage the wellbeing of a patient prior to reaching a critical pressure value.

Efforts have been underway for years to develop pressure sensors for temporary or chronic use in a body organ or vessel, including those relating to the measurement or monitoring of intracranial fluid pressure. Many different designs and operating systems have been proposed and placed into temporary or chronic use with patients.

Many pressure measurements need to be gage referenced to barometric pressure and independent of barometric pressure. Atmospheric pressure can vary for a number of reasons. Altitude is probably the most significant cause of atmospheric pressure variation. Atmospheric pressure declines by approximately 0.5 psi per 1000 feet increase in altitude. Weather is another cause of atmospheric pressure variation. At a given location, weather-induced atmospheric pressure variation can be on the order of 20 mm Hg (0.1 psi).

In order to measure a pressure independent of barometric pressure pertaining to a living patient, existing solutions include a single differential pressure sensor where one side of a pressure sensing membrane has an unknown pressure applied, while the other side of the membrane has atmospheric pressure applied. In this configuration, the deflection of the membrane is a direct function of the difference between the unknown pressure applied and atmospheric pressure. This enables the unknown pressure to be sensed. However, when the unknown pressure is remote such as inside a living body at the distal end of a long catheter that exits the body, there is a need to connect one side of the membrane to atmospheric pressure and this is done by an open cavity running the length of the catheter and terminating at the proximal end. If the open cavity is filled with fluid, then the differential pressure sensor can be located at the proximal end of the catheter. However, the differential pressure measured will be affected by the head of pressure induced by the fluid in the catheter, and this head will be a function of the difference in height between the opening at the distal end of the catheter and the height of the pressure sensor. Furthermore, the capacity to measure quickly changing pressures (such as blood pressure) will be compromised by the presence of the fluid which forms a low pass filter. A more accurate pressure measurement is obtained by placing the sensor at the distal end of the catheter. This is how blood and intracranial pressures are measured conventionally when using a catheter tipped sensor.

A single differential pressure sensor needs an open lumen down the length of the catheter to accommodate air to conduce pressure between one side of a pressure sensing membrane having an unknown pressure applied and the other side of the membrane having atmospheric pressure applied. When a catheter is put into a human body the materials will start to absorb moisture, and when sufficient moisture migrates into the open lumen and condenses, then the lumen can become blocked and the ability to measure differential pressures accurately is lost. It can also be difficult to detect that the sensitivity or offset of the pressure measurement is being compromised by condensation inside the lumen. A lumen is also sensitive to closure if the catheter is bent which will cause a measurement failure.

An alternative approach is to make the sensor fully implanted and use a wireless communication technique to relay the pressure to the exterior. For an implantable device, there is not an option to connect the membrane to atmospheric pressure because the system is fully under the skin (an exit site would be a risk for infection). In these circumstances, an absolute pressure sensor is used where one side of the membrane is connected to a vacuum to create a pressure sensor independent of atmospheric pressure. As atmospheric pressure varies, the absolute pressure sensor output will also vary, however this component of the signal is not related to the unknown pressure to be sensed. Hence an independent measure of atmospheric pressure is taken so that it can be subtracted from the absolute pressure measurement to derive the unknown pressure to be sensed. An example is the TRM54P (Millar Inc.) implantable pressure sensor system where an absolute pressure sensor is used in the implantable system which transmits the absolute pressure to an external receiver. The external receiver TR181 (Millar Inc) includes an absolute pressure sensor to measure atmospheric pressure and, using an algorithm on a microprocessor, subtracts the atmospheric pressure sensor from the TRM54P signal and reports the pressure measured as a difference from atmospheric pressure.

To those practiced at measuring pressure, it would seem obvious that to measure a differential pressure, one should choose to use a single differential pressure sensor over a pair of absolute pressure sensors. A single differential pressure sensor has advantages such as fewer components, the components do not require a vacuum to be maintained after manufacture, and the sensor performance in terms of drift and span is more likely to be superior than a pair of absolute sensors. However, when the application of differential pressure measurement relies on a vented lumen along the length of the catheter, a single differential sensor can be unreliable. Alternatively, the design and cost of manufacture of the catheter can be sub-optimal as it needs to be larger to include the open lumen channel. A catheter without an open lumen is likely to be smaller and more reliable.

It would be advantageous to have a pressure sensor that does not depend on the vented lumen the length of the catheter.

### SUMMARY

An embodiment of the disclosure is a differential pressure sensor system with at least two absolute pressure sensors comprising an external absolute pressure sensor with a pressure sensitive surface in contact with atmospheric pressure; at least one internal absolute pressure sensor, each internal absolute pressure sensor with a pressure sensitive surface in contact with one or more regions at an unknown pressure; and a means to calculate a difference between the external sensor and at least one internal absolute pressure sensor to derive the pressure in one or more regions. In an embodiment, the at least one internal absolute pressure sensor is located along the length of a catheter away from the proximal end of the catheter, and the external absolute pressure sensor is located at or near the proximal end of the catheter. In an embodiment, the catheter is filled with a filler material. In an embodiment, a pressure signal derived from the external absolute pressure sensor is subtracted from pressure signals from each internal absolute pressure sensor and a result is interpreted as the differential pressure of each region with respect to atmospheric pressure. In an embodiment, the external absolute pressure sensor and the at least one internal absolute pressure sensor are a piezo-resistive MEMs sensor. In an embodiment, each absolute pressure sensor is part of a Wheatstone bridge circuit; a voltage output from the Wheatstone bridge circuit for the external absolute pressure sensor is connected to a first input of a differential amplifier; a voltage output from a second Wheatstone bridge circuit for the internal absolute pressure sensor is connected to a second input of the differential amplifier; and the output of the differential amplifier is interpreted as a differential pressure. In an embodiment, an electrical circuit that derives the differential pressure is located at the proximal end of the catheter. In an embodiment, the differential pressure sensor system further comprises a temperature compensation circuit and offset compensation circuit. In an embodiment, the output voltage is normalized to 5 microVolts per Volt of excitation per mmHg In an embodiment, the temperature of each absolute pressure sensor is measured and a pressure measurement is adjusted. In an embodiment, the absolute pressure sensor is a capacitive pressure sensor. In an embodiment, the absolute pressure sensor includes a digital interface compatible with a digital microprocessor. In an embodiment, the digital microprocessor computes a difference between the absolute pressure measurements. In an embodiment, the digital microprocessor computes a pressure compensation based on the measurement of temperature from the sensors. In an embodiment, the absolute pressure sensor is an optical pressure sensor. In an embodiment, the absolute pressure sensor is a half bridge pressure sensor.

An embodiment of the disclosure is a method of deriving a pressure in one or more regions comprising using the differential pressure sensor system.

An embodiment of the disclosure is a pressure system with at least two absolute pressure sensors comprising an external absolute pressure sensor with a pressure sensitive surface in contact with atmospheric pressure; at least one internal absolute pressure sensor, each internal absolute pressure sensor with a pressure sensitive surface in contact with one or more regions at an unknown pressure; and a means to calculate a difference between the external absolute pressure sensor and at least one internal absolute pressure sensor to derive the pressure in one or more regions.

In an embodiment, the at least one internal absolute pressure sensor is located along the length of a catheter at or near the distal end of a catheter, and the external absolute pressure sensor is located at or near the proximal end of the catheter and outside the catheter. The catheter allows communication of the pressure (represented as a voltage or other signal) via wires to the exterior, as well as providing a means of introducing the internal sensor to the measurement location.

In an embodiment, the pressure signal derived from the external absolute pressure sensor is subtracted from the pressure signals from each internal absolute pressure sensor and a result is interpreted as the differential pressure of each region with respect to atmospheric pressure. In an embodiment, the external absolute pressure sensor and the at least one internal absolute pressure sensor are a piezo-resistive MEMs sensor. In an embodiment, each absolute pressure sensor is part of a Wheatstone bridge circuit; a voltage output from the Wheatstone bridge circuit for the external absolute pressure sensor is connected to a negative input of a differential amplifier; a voltage output from a second Wheatstone bridge circuit for a second absolute pressure sensor is connected to the positive input of a differential amplifier; and the output of the differential amplifier is interpreted as a differential pressure. In an embodiment, an electrical circuit that derives the differential pressure is located at the proximal end of the catheter and outside the catheter. In an embodiment, a housing around the electrical circuit is a connector system compatible with existing differential pressure sensor catheters. In an embodiment, the output voltage is normalized to 5 microVolts per Volt of excitation per mmHg

In one embodiment, the internal sensor is a half bridge sensor which is smaller than the conventional full bridge sensor as it has fewer sense resistors in order to make the sensor as small as possible. As an alternative to two independent full bridge circuits, the two half bridge sensors can be combined to form a single full bridge circuit.

In an embodiment, the temperature of each absolute pressure sensor is measured and a pressure measurement is adjusted. In an embodiment, the absolute pressure sensor is a capacitive pressure sensor. In an embodiment, the absolute pressure sensor includes a digital interface compatible with a digital microprocessor. In an embodiment, the digital microprocessor computes a difference between the absolute pressure measurements. In an embodiment, the digital microprocessor computes a pressure compensation based on the measurement of temperature from the sensors. In an embodiment, the microprocessor is connected to a radio system capable of transmitting and receiving data with a remote monitoring system.

The foregoing has outlined rather broadly the features of the present disclosure in order that the detailed description that follows can be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 is a bridge circuit in accordance with the described embodiments;

FIG. 2 is a cross-section view of a catheter tip measurement device in accordance with the described embodiments;

FIG. 3 is a block diagram of a differential pressure sensor system with two absolute pressure sensors in accordance with the described embodiments;

FIG. 4 is a schematic diagram of the differential pressure sensor system of FIG. 3.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure can be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3rd Edition.

As used herein, the term “distal” means and refers to situated away from the point of attachment or origin.

As used herein, the term “proximal” means and refers to next to or nearest the point of attachment or origin.

The device can determine the pressure between the measurement site (e.g., the brain) and the exterior of the patient by using a pair of absolute pressure sensors electrically connected by a catheter. In an embodiment, an absolute sensor has a diaphragm with strain gages on it which forms part of a vacuum cavity. This means the recorded pressure is referenced to a vacuum. The catheter allows communication of the pressure (represented as a voltage or other signal) via wires to the exterior, as well as providing a means of introducing the internal sensor to the measurement location. At the exterior side is another absolute pressure sensor which records the barometric pressure. Subtracting the barometric pressure from the absolute internal pressure gives the differential pressure (between the site where the sensor is located in tissue and the atmosphere) which is the pressure of interest clinically.

In an embodiment, two sealed absolute pressure sensors can be used to measure differential pressure. In an embodiment, the internal absolute sensor is located at the distal end of a catheter and the external absolute sensor is located at the proximal end of the catheter. A differential pressure signal is derived from the pressure difference between the two absolute pressure sensors.

A pressure system with at least two absolute pressure sensors can have an external sensor with a pressure sensitive surface in contact with atmospheric pressure (proximal) and internal sensors each with a pressure sensitive surface in contact with one or more regions at an unknown pressure (distal). In an embodiment, there are three absolute pressure sensors. In an embodiment, there are four absolute pressure sensors. In an embodiment, there are five or more absolute pressure sensors. The unknown pressure is determined by a means to calculate the difference between the external sensor and the internal sensors. The two sealed absolute pressure sensors allow direct replacement of differential sensor catheters because a standard electrical interface can be provided. The two sealed absolute pressure sensors are an advantage over use of one differential sensor because of increased reliability and removing the need for an open lumen inside the catheter.

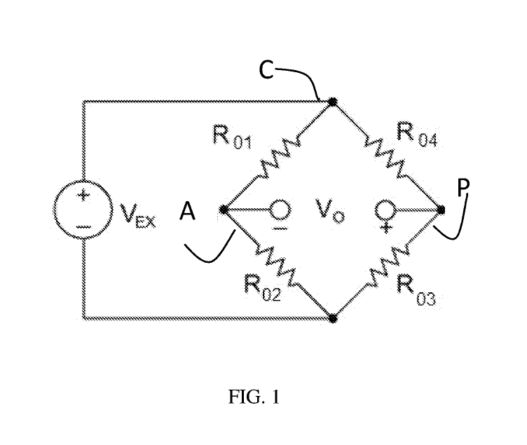

In an embodiment, the absolute pressure sensor is part of a Wheatstone bridge circuit. the internal sensor is a half bridge sensor which is smaller than the conventional full bridge sensor as it has fewer sense resistors in order to make the sensor as small as possible. A general bridge circuit is shown in FIG. 1. A full bridge is made up of four resistors. A half-bridge sensor will have two resistors, and these might be labeled R01 and R04 in FIG. 1. With an increase in pressure, one of the resistors (R01) will increase resistance and the second resistor (R04) will decrease resistance. In one embodiment, the resistors R02 and R03 are discrete resistors and these are located at the proximal end. In one embodiment, each absolute sensor has discrete resistors to make up two independent full bridge circuits. Each full bridge circuit produces an output voltage, and these voltages are subtracted to obtain a differential pressure signal. The subtraction can be done using analog circuits, or the bridge output voltages can be converted to digital signals and the subtraction performed using an algorithm on a microprocessor.

As an alternative to two independent full bridge circuits, the two half bridge sensors can be combined to form a single full bridge circuit. In this configuration, with an increase in the unknown pressure R01 resistance will increase, R04 resistance will decrease, and with an increase in atmospheric pressure, R02 resistance will increase and R03 resistance will decrease. The components are designed to have the same sensitivity to pressure change, so if the unknown pressure increases by the same amount as the atmospheric pressure, then the voltage at the junction of R01 and R02 (node A) will not change and the voltage at the junction of R3 and R4 (node P) will not change, and the difference output voltage Vo remains unchanged (because the difference between the unknown pressure and atmospheric pressure is unchanged). Combining the internal sensors to form a full bridge sensor has the benefits of smaller offset, matched output sensitivity and improved linearity. This improves the performance and reduces the calibration cost of the sensor.

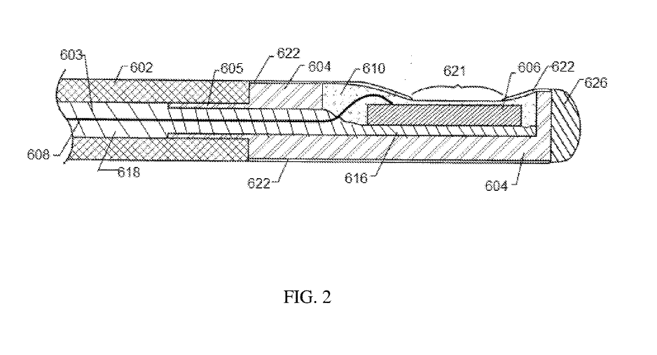

FIG. 2 depicts a side cross-section view of a catheter tip measurement device according to the present invention. Referring to FIG. 2, a window 621 is cut out of a tubular metal casing 604 and an internal absolute sensor 606 is located at the window. The tubular metal casing 604 can be attached to a catheter 602 by inserting an annular connecting portion 605 into the end of catheter 602. The annular connecting portion 605 can be created by machining the proximal end of a tubular metal casing.

An epoxy bead 626 can be placed at the distal end of the device, as shown in FIG. 2, to close the end of the casing 604 and to provide smooth entry characteristics for the catheter tip measurement device. Other measurement devices can be attached to the distal tip of the device. For example, a thermistor measurement device could be attached to the distal tip of casing 604. Such additional measurement devices would provide dual measurement capabilities with pressure sensor 606.

To insulate the internal absolute sensor 606, a flexible insulating material 610 can be applied on top of the pressure sensor 606, as shown in FIG. 2. As with prior devices, this material can be flexible room-temperature-vulcanizing (RTV) silicone rubber, which is slightly tacky. To insulate the device from body tissues, an insulating layer 622 is applied to surround the measuring tip of the device. In an embodiment, the insulating layer 622 is an epoxy resin coating which will effectively insulate the internal parts of the device from the outside world.

A window 621 is provided in the insulating layer 222 over the sensing diaphragm region of the internal absolute sensor 606, a layer of flexible RTV silicone rubber is placed over the pressure sensing diaphragm. Generally, the thickness of RTV silicone rubber placed over the pressure sensing diaphragm of the pressure sensor 606 is approximately 100 μm, providing an insulation strength of approximately 600-800 volts. Signal wires 608 are isolated and extend from the internal absolute sensor 606 through internal channel 618 of the catheter 602 to outside of the catheter 602, such that a pin-compatible solution to a conventional differential pressure signal catheter is produced. To insulate the absolute sensor 606 from the inner of the tubular metal casing 604, a gap 616 is provided between the sensor 606 and the inner of the tubular metal casing 604.

A catheter tip measurement device has been used in prior devices, such as a device disclosed in U.S. Pat. No. 5,902,248. However, in prior devices, there must be an open lumen from outside access to the back of the internal sensor. The venting channel must generally be of a sufficient size to equalize the reference side of strain gauge diaphragm of the pressure sensor to the reference pressure. An opening of approximately 0.002 inches or more in diameter is generally required to achieve this venting requirement.

In the present disclosure, the catheter 602 can be lower cost to manufacture because it does not need to include an open lumen, smaller in diameter, and provide longer operation. The catheter 602 is used to accommodate the absolute pressure sensor 606 and signal wires 608. Since there is no need to provide an air way to the back of the sensor to reference it to atmosphere, the mounting of the sensor into the catheter is simpler and manufacturing cost can be reduced. In one embodiment, the catheter does not need to be hollow and mechanical properties can be improved since more of the structure can be dedicated for the purpose of electrical connection. In one embodiment, the wires are structurally stronger and occupy the space that was previously used for the air-filled lumen providing better durability for the same size catheter diameter. In an embodiment, it also can improve the durability of the wires by allowing the use of a filler material 603, such as CF19-2186 (Avantor Inc.) in the internal channel 618 and the gap 616 to prevent condensation which could cause corrosion and failure of the communication wires. The device overcomes a vulnerability of the open lumen developing condensation which blocks the lumen. This device has the prospect of long use in the body and a smaller dimensional diameter (a small open lumen is more vulnerable to condensation than a larger open lumen, so as the catheter gets smaller, the benefits of not needing a lumen are higher. As detailed above, a lumen in a single differential pressure sensor is sensitive to closure if the catheter is bent. Electrical connections in the present disclosure can be vulnerable to repetitive bending, but bending will not cause an immediate failure.

In an embodiment, the absolute pressure sensors can be used in a catheter to measure pressure associated with an intravascular microaxial blood pump. In an embodiment, the absolute pressure sensors can be used in, including but not limited to, cardiovascular, ablation, research, respiratory, intracranial, body cavity, and urological/rectal applications.

Catheter design with two absolute pressure sensors is a benefit over the use one differential sensor.

In an embodiment, compensation electronics to implement null offset, and temperature sensitivity can be included.

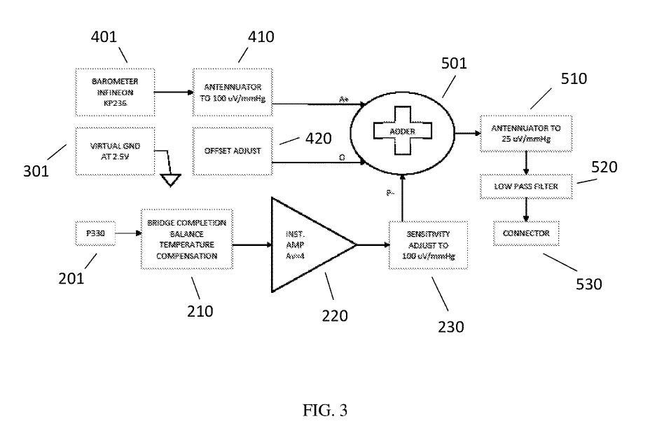

FIG. 3 is a simplified block diagram of a differential pressure sensor system with two absolute pressure sensors. An absolute pressure sensor 201 is located along the length of a catheter (not shown) at or near the distal end of the catheter which can be inserted within a body portion where a pressure measurement is required. An absolute pressure sensor 401 contacts with atmosphere. The absolute pressure sensors 201 and 401 can be of including but not limited to piezo-resistive, piezo-electric, capacitive, or optical type.

In an embodiment, the absolute pressure sensor 201 is a P330 series piezo-resistive pressure die. In an embodiment, the P330 is manufactured by NovaSensor's proprietary SenStable® that performs absolute pressure sensing and has excellent measurement accuracy. Piezo-resistive pressure sensors are one of the products of MEMS technology. The sensing material in a piezo-resistive pressure sensor is a diaphragm formed on a silicon substrate, which bends with applied pressure. A deformation occurs in the crystal lattice of the diaphragm because of that bending. This deformation causes a change in the band structure of the piezo-resistors that are placed on the diaphragm, leading to a change in the resistivity of the material. This change can be an increase or a decrease according to the orientation of the resistors. A circuit in the sensor is used to transform the change of resistivity into an electrical signal that is proportional to the applied pressure. In an embodiment, the P330 has a diaphragm with strain gages on it which forms part of a vacuum cavity which means the recorded pressure is referenced to a vacuum and the P330 is an absolute pressure sensor. The P330 employs a Wheatstone half-bridge design which requires two external resistors to complete a full-bridge configuration. When excited with a DC voltage source, the P330 produces a mV output that is proportional to applied pressure. Because the change in resistance is so small, a high gain amplifier is required to amplify the resistance-related voltage change. The P330 can withstand a standard pressure range of 450-1050 mmHgA and a 4500 mmHgA burst pressure. The drift characteristics of the P330 used for rat telemeters have shown consistent drift performance of less than plus or minus 1.5 mmHg over 2 weeks. In an embodiment, electronics are used to subtract the atmospheric sensor output from the P330 pressure sensor output.

In an embodiment, the absolute pressure sensor 401 is an analog absolute pressure sensor KP236 manufactured by Infineon®. The KP236 is a miniaturized analog barometric sensor IC based on a capacitive principle. The calibrated transfer function converts a pressure range of 40 KPa to 115 KPa into a voltage range of 0.5V to 4.5V. The pressure is detected by an array of capacitive surface micromachined sensor cells. The sensor cell output is amplified, temperature compensated and linearized to obtain an output voltage that is proportional to the atmospheric pressure. All parameters needed for the complete calibration algorithm such as offset, gain, temperature coefficients of offset and gain, and linearization parameters are determined after assembly.

The signal of the absolute pressure sensor 201 is transmitted through signal wires along the catheter to a bridge completion and temperature compensation module 210. The module 210 completes a four-arm Wheatstone bridge with an excitation voltage. The output voltage of the compensation module 210 is amplified by an amplifier 220 to a level that can be adjust to 100 uV/mmHg by a sensitivity adjust circuit 230.

Referring back to FIG. 2, signal wires 608 are inside the catheter 602, the gap around the wires needs to be just large enough to allow the wires to be strung down the tube, and the gap can be filled afterwards to avoid any area of condensation.

In one embodiment, the voltage signal of the absolute pressure sensor 401, which measures atmospheric pressure, is transmitted to an attenuator 410 to attenuate the voltage signal to 100 uV/mmHg The voltage signal from a P330 absolute pressure sensor 201 provides the input to a bridge completion balance circuit 210 which is then amplified by 220, and sensitivity adjusted by 230. At this stage, the two processed pressure signals can be subtracted using the adder 501. An offset adjustment is available from 420. The output of the adder is normalized to a common output sensitivity using 510, this signal is low pass filtered 520 to reduce any high frequency noise. A connector 530 is compatible with the input stage of a patient monitor.

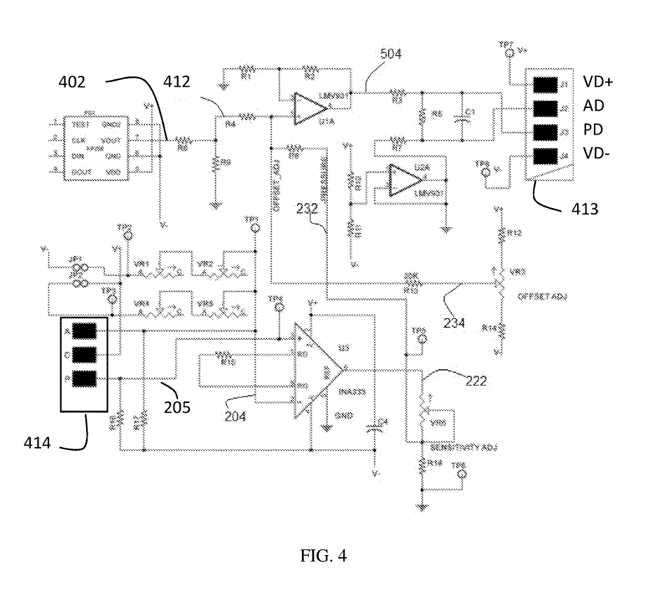

The output at connector 530 has the same characteristics as a conventional differential pressure catheter, and the four contacts are shown in 413 FIG. 4. This means that the two absolute pressure sensor catheter can be used as a direct replacement for a single differential pressure sensor catheter.

The accuracy or consistency of the pressure sensor measurements depends on certain properties, parameters, characteristics, or conditions ideally remaining substantially unchanged. Unfortunately, it is impossible to ideally maintain such constant parameters. Therefore, over time pressure sensors undesirably exhibit a drift in pressure measurements due to unwanted mechanical stress.

In an embodiment, the differential pressure sensor system includes an offset adjust circuit 420 to compensate for residual voltages at the sensor output caused by manufacturing tolerances. The voltage signal generated by the offset adjust circuit 420 is input into the adder 501, and the output voltage of the adder 501 is proportional to the difference between the sum of voltage signal from attenuator 410 and voltage signal generated by the offset adjust circuit 420 and the voltage signal from sensitivity adjust circuit 230.

The output voltage of the adder 501 is attenuated to 25 uV/mmHg by an attenuator 510 and then filtered by a low pass filter 520 to filter out noise. The filtered voltage indicates the pressure of the system. It can be measured by voltage measuring equipment or it can be input into an A/D module which connects with a microprocessor. The microprocessor can read digital data of the voltage from the A/D module and calculate pressure based on the voltage. In an embodiment, the system can include a displayer connected to the microprocessor to display results of the pressure measurement. In another embodiment, the microprocessor is connected to a radio system capable of transmitting and receiving data with a remote monitoring system. In an embodiment, the microprocessor connects to a controller of an intravascular microaxial blood pump to feedback the signal of the pressure such that the blood pump regulates the volume flow and the pressure.

In an embodiment, the power supply voltage is 5 V, in order to maximize the dynamic range of the amplifier, a virtual ground 301 is set to be one-half of the supply voltage at 2.5 V.

FIG. 4 is a schematic diagram of the differential pressure sensor system of FIG. 3. The absolute pressure sensor 201 in FIG. 3 is a P330 series piezo-resistive pressure die and connects to the circuit at 414. The P330 employs a Wheatstone half-bridge design which requires two external resistors to complete a full-bridge configuration. In FIG. 4, the power supply is V⁺ and V⁻. In one embodiment, V⁺ is 5 V and V⁻ is 0 V. Resistors R16 and R17 connect to the half-bridge of P330 (not shown) to complete a full-bridge configuration. In one embodiment, the resistances of R16 and R17 are both 3.16 KΩ. The output voltage 204 of one side of the Wheatstone full-bridge is connected to a negative input of an amplifier INA333. The output voltage 205 of the other side of the Wheatstone full-bridge is connected to a positive input of an amplifier INA333. In an embodiment, the amplifier is manufactured by Texas Instruments. A resistance R15 is coupled between pins 1 and 8 of the INA333. In one embodiment, the resistance of R15 is 33.2KΩ and according to the gain equation of INA333: G=1+100 KΩ/33.2KΩ=4. V⁺ and V⁻ are supplied to the INA333. In this case, a positive increase in pressure at sensor 201 will generate a larger negative voltage output voltage 222 of the INA333. Output 222 is inverted by the INA333 to generate the input to the adder section which achieves a subtraction from atmospheric pressure.

In an embodiment, the output voltage 222 of the INA333 is applied to a sensitivity adjust circuit composited of series variable resistance VR6 and resistance R18 which consist of a voltage divider. In one embodiment, the resistance of VR6 is 1 KΩ and the resistance of R18 is 510Ω. Through the sensitivity adjust circuit, the output voltage 222 can be adjust to a voltage signal 232 indicated of 100 uV/mmHg.

In FIG. 4, in an embodiment, A, C, and P are three connections 414 to the P330 sensor. In an embodiment, A, C, and P come from the P330 sensor at the catheter tip. In an embodiment, VD⁺, AD, PD, and VD⁻ are outputs from this circuit and form a connector 413. 413 is compatible with a patient monitor.

In FIG. 4, an analog absolute pressure sensor KP236 converts the atmospheric pressure into a voltage. KP236 includes a calibration and temperature compensation circuit. The output voltage 402 of KP236 is applied to an attenuator. The attenuator is a voltage divider composited of series resistance R6 and R9. In one embodiment, the resistances of R6 and R9 are 100 KΩ and 1.43 KΩ respectively. Through the attenuator, the output voltage 402 can be attenuated to a voltage signal 412 indicating 100 uV/mmHg.

An offset adjust circuit is provided in FIG. 4, the offset adjust circuit consists of series connected resistance R12, VR3 and R14 spanned between the power supply V⁺ and V⁻ to form a voltage divider. The output voltage 234 of the offset adjust circuit is the voltage on the tap of the VR3, when the resistance value of VR3 changes, the output voltage 234 of the offset adjust circuit will change accordingly.

The voltage signal 232, the voltage signal 412 and the output voltage 234 couple to a positive input of an amplifier U1A through resistances R8, R4, and R13 respectively. In one embodiment, the amplifier U1A is LMV931 manufactured by Texas Instruments, the resistance values of R8, R4, and R13 are 20 KΩ. The positive input of the amplifier UTA couples to one end of a resistance R1 and one end of a resistance R2, the other end of the resistance R1 is grounded while the other end of the resistance R2 couples to the output of the amplifier U1A. therefore, the output voltage 504 of the amplifier U1A=⅔(the voltage signal 412+the output voltage 234+the voltage signal 232). The voltage signal 232 is an inverted representation of the pressure at 201.

The output voltage 504 of the amplifier U1A is then applied to a RC network and an amplifier U2A through a resistance R3, wherein a resistance R5 and a capacitor C1 consist of a low pass filter to pass the signal with frequencies less than 4 kHz. In one embodiment, the resistance R5 is 1.62 KΩ and the capacitor C1 is 0.39 μF. A voltage divider composed of R10 and R11 spanned between V⁺ and V⁻ provides a voltage to a positive input of an amplifier U2A, the amplifier U2A is constructed as a voltage follower and provides a voltage to the RC network through a resistance R7. In an embodiment, the resistances R3, R7, R10, R11 are 1.33 KΩ, 1.33 KΩ, 20 KΩ and 20 KΩ respectively and the amplifier U2A is also LMV931. In an embodiment, the output voltage 504 can be attenuated down to 25 uV/mmHg and the signal impedance can be set to 1 KΩ.

In an embodiment, in order to calibrate the differential pressure sensor system, two bridge jumpers JP1 and JP2 are set open on the circuit of the system to isolate trimming resistors VR1, VR2, VR4 and VR5. Test points TP1, TP4 and TP6 are used to provide access to a precision digital multimeter (DMM) to measure the voltage from the sensor at two temperatures. Compensation resistance values are then calculated. Using TP1 and TP3, the values of VR4 and VR5 are set to match the correct compensation resistance. Using TP2 and TP1 the values of VR1 and VR2 are set to match the correct compensation resistance. When the jumpers are closed, the circuit consisting of VR1, VR2, VR4 and VR5 will provide temperature compensation for the output signal of the INA333 amplifier.

As described above, in clinical practice, measurement of pressure requires a small diameter catheter with a high-fidelity sensing element. The device uses a piezo-resistive pressure die to generate signal proportional to the pressure and wires to communicate signal out of the catheter. In an embodiment, there is no need for wireless equipment and power in the catheter needed in prior art implanted differential pressure sensor systems. Therefore, the catheter can have smaller diameter than in prior art.

In other implementations, such as an optical sensor, the catheter can be a solid optical fiber which is smaller than a catheter which includes a hollow lumen.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations can be applied to the compositions and methods and in the steps or in the sequence of steps of the methods described herein without departing from the concept, spirit and scope of the disclosure. More specifically, it will be apparent that certain agents which are related can be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A differential pressure sensor system with at least two absolute pressure sensors comprising
an external absolute pressure sensor with a pressure sensitive surface in contact with atmospheric pressure;
at least one internal absolute pressure sensor, each internal absolute pressure sensor with a pressure sensitive surface in contact with one or more regions at an unknown pressure; and
a means to calculate a difference between the external sensor and at least one internal absolute pressure sensor to derive the pressure in one or more regions.

2. The differential pressure sensor system of claim 1 wherein the at least one internal absolute pressure sensor is located along the length of a catheter away from the proximal end of the catheter, and the external absolute pressure sensor is located at or near the proximal end of the catheter.

3. The differential pressure sensor system of claim 2, wherein the catheter is filled with a filler material.

4. The differential pressure sensor system of claim 1 wherein a pressure signal derived from the external absolute pressure sensor is subtracted from pressure signals from each internal absolute pressure sensor and a result is interpreted as the differential pressure of each region with respect to atmospheric pressure.

5. The differential pressure sensor system of claim 1 wherein the external absolute pressure sensor and the at least one internal absolute pressure sensor are a piezo-resistive MEMs sensor.

6. The differential pressure sensor system of claim 5 wherein each absolute pressure sensor is part of a Wheatstone bridge circuit;
a voltage output from the Wheatstone bridge circuit for the external absolute pressure sensor is connected to a first input of a differential amplifier;
a voltage output from a second Wheatstone bridge circuit for the internal absolute pressure sensor is connected to a second input of the differential amplifier; and
the output of the differential amplifier is interpreted as a differential pressure.

7. The pressure sensor system of claim 6 where an electrical circuit that derives the differential pressure is located at the proximal end of the catheter.

8. The differential pressure sensor system of claim 6, further comprising a temperature compensation circuit and offset compensation circuit.

9. The differential pressure sensor system of claim 6 wherein the output voltage is normalized to 5 microVolts per Volt of excitation per mmHg

10. The differential pressure sensor system of claim 1 wherein the temperature of each absolute pressure sensor is measured and a pressure measurement is adjusted.

11. The differential pressure sensor system of claim 1 wherein the absolute pressure sensor is a capacitive pressure sensor.

12. The differential pressure sensor system of claim 1 wherein the absolute pressure sensor includes a digital interface compatible with a digital microprocessor.

13. The differential pressure sensor system of claim 12 wherein the digital microprocessor computes a difference between the absolute pressure measurements.

14. The differential pressure sensor system of claim 13 wherein the digital microprocessor computes a pressure compensation based on the measurement of temperature from the sensors.

15. The differential pressure sensor system of claim 1 wherein the absolute pressure sensor is an optical pressure sensor.

16. The differential pressure sensor system of claim 1 wherein the absolute pressure sensor is a half bridge pressure sensor.

17. A method of deriving a pressure in one or more regions comprising using the differential pressure sensor system of claim 1.

18. A method of deriving a pressure in one or more regions comprising using the differential pressure sensor system of claim 2.

19. A method of deriving a pressure in one or more regions comprising using the differential pressure sensor system of claim 4.

20. A method of deriving a pressure in one or more regions comprising using the differential pressure sensor system of claim 6.
